(19) United States
(12) Patent Application Publication
(10) Pub. No.: US 2023/0149542 A1
(43) Pub. Date: May 18, 2023

(54) NUTRITIONAL FORMULATIONS FOR MODULATING RESPIRATORY-INDUCED CYTOKINES

(71) Applicant: ABBOTT LABORATORIES, Abbott Park, IL (US)

(72) Inventors: RACHAEL BUCK, Gahanna, OH (US); MICHAEL FARROW, Bexley, OH (US)

(21) Appl. No.: 17/917,381

(22) PCT Filed: Apr. 6, 2021
(86) PCT No.: PCT/US2021/025945

§ 371 Date: Oct. 6, 2022

Related U.S. Application Data
(60) Provisional application No. 63/005,656, filed on Apr. 6, 2020.

(51) Int. Cl. A61K 39/395; A61K 47/12; A61K 47/10; A61P 29/00; A61P 11/00; A61P 31/12; A23L 33/00; A23L 33/12; A23L 33/125; A23L 33/19; A23L 33/155

(52) U.S. Cl. CPC A61K 39/395; A61K 47/12; A61K 47/10; A61P 29/00; A61P 11/00; A61P 31/12; A23L 33/40; A23L 33/12; A23L 33/125; A23L 33/19; A23L 33/155

(57) ABSTRACT

Disclosed are compositions, including nutritional compositions, for reducing illness/infection related symptoms. In particular, administration of the inventive compositions helps to reduce symptoms such as those selected from impaired cognition, fever, anhedonia, loss of appetite, hyperalgesia, and sleep disturbances, lethargy, chills, irritability, and skin hypersensitivity to touch that often result from respiratory virus-induced inflammation.

### CROSS-REFERENCE TO RELATED APPLICATIONS

This application claims priority to and the benefit of U.S. Provisional Patent Application No. 63/005,656, filed on Apr. 6, 2020, the entire content of which is incorporated herein by reference.

### FIELD

The present disclosure relates to a composition for improving one or more aspects of the individual's health. More particularly, the present disclosure relates to compositions, including nutritional compositions, comprising an ingredient to modulate or improve the immune response of an individual consumer by reducing or preventing one or more symptoms of a particular condition or disease as described in greater detail herein.

### BACKGROUND

The body's natural response to infections and infectious agents is to marshal the immune system, including increasing antibody activity and cytokine expression. Increases in pro-inflammatory cytokines often promote symptoms and behaviors associated with an infection, but which do not necessarily aid the body in fighting or eliminating the infection. The symptoms include fever, sleep disorders, increased pain (response), impaired cognition, anhedonia (mood affect, malaise), and atypical appetite or loss of appetite. These undesirable symptoms can prolong recovery and/or worsen the experience associated with the infection.

Despite recognizing that many of these by-products of infection response are unnecessary, efforts to develop a therapy that can overcome this issue have yet to be fully developed. Accordingly, there is an unmet need for improved compositions that are effective to treat one or more of the conditions described herein.

### SUMMARY

The general inventive concepts are directed to compositions and methods including a natural bovine immunoglobulin, alone or in combination with another functional ingredient, for use in modulating or treating the symptoms/conditions or diseases described herein, including but not limited to: infectious disease-related behavioral symptoms and/or morbidity. In particular, the symptoms include: impaired cognition, fever, anhedonia, loss of appetite, hyperalgesia, sleep disturbances, lethargy, chills, irritability, and skin hypersensitivity to touch. In certain exemplary embodiments, the general inventive concepts contemplate a method to decrease cytokine burden and ameliorate sickness/illness behavior symptoms by reducing respiratory virus-induced inflammation. In certain embodiments, the general inventive concepts contemplate a nutritional method to decrease cytokine burden and ameliorate sickness/illness behavior symptoms by reducing respiratory virus-induced inflammation.

In certain exemplary embodiments, the general inventive concepts relate to a composition comprising a bovine immunoglobulin for modulating an infectious agent-induced cytokine response.

In certain exemplary embodiments, the general inventive concepts relate to a method of modulating respiratory virus-induced inflammation in an individual infected by a respiratory virus. The method comprises administering a composition comprising from 0.01 g/L to 100 g/L of bovine immunoglobulin to the individual.

In certain exemplary embodiments, the general inventive concepts relate to a composition comprising from 0.01 g/L to 100 g/L of bovine immunoglobulin for use in a method of ameliorating and/or treating infectious disease-related behavioral symptoms and/or morbidity. The symptoms include: impaired cognition, fever, anhedonia, loss of appetite, hyperalgesia, sleep disturbances, lethargy, chills, irritability, and skin hypersensitivity to touch.

In certain exemplary embodiments, the general inventive concepts relate to method of treating respiratory virus-induced symptoms by administering a composition, the composition comprising from 0.01 g/L to 100 g/L of bovine immunoglobulin.

In certain exemplary embodiments, the general inventive concepts relate to a method of modulating excessive inflammatory cytokine burden, the method comprising administering a composition comprising from 0.01 g/L to 100 g/L of bovine immunoglobulin to the individual.

### BRIEF DESCRIPTION OF THE DRAWINGS

The general inventive concepts, as well as embodiments and advantages thereof, are described below in greater detail, by way of example, with reference to the drawings in which:

FIG. 1 is a bar graph of results for administration of bovine immunoglobulin on RSV.

FIG. 2 is a bar graph of results for administration of bovine immunoglobulin on hAdV.

FIG. 3 is a bar graph of results for administration of bovine immunoglobulin on hRV.

FIG. 4 is a picture of a cell culture showing effects of exposure to bovine immunoglobulin.

FIG. 5 is a bar graph showing the amount of IFN-g after exposure to various viruses.

FIG. 6 is a bar graph showing the amount of IL-6 after exposure to various viruses.

FIG. 7 is a bar graph showing the amount of IL-1B after exposure to various viruses.

FIG. 8 is a bar graph showing the amount of IL-8 after exposure to various viruses.

FIG. 9 is a bar graph showing the amount of change for various markers versus RSV infected control.

FIG. 10 is a bar graph showing the amount of change for various markers versus RV16 infected control.

### DETAILED DESCRIPTION

The compositions and methods described herein utilize bovine immunoglobulin alone or in combination with another functional ingredient, for ameliorating and/or treating a number of diseases and conditions as described in greater detail herein. The compositions, including nutritional compositions, described herein include synthetic formulas that comprise bovine immunoglobulin. These and other features of the compositions and methods, as well as some of the many optional variations and additions, are described in detail hereafter.

The terms “nutritional formulation” or “nutritional composition” as used herein, are used interchangeably and, unless otherwise specified, refer to synthetic formulas including nutritional liquids, nutritional powders, nutritional solids, nutritional semi-solids, nutritional semi-liquids, nutritional supplements, and any other nutritional food product as known in the art. The nutritional powders may be reconstituted to form a nutritional liquid, all of which comprise one or more of fat, protein and carbohydrate and are suitable for oral consumption by a human and may be used as a sole or supplemental source of nutrition. The terms “nutritional formulation” or “nutritional composition” do not include human breast milk and do not refer to supplemented milk and are generally shelf stable.

The term “nutritional liquid” as used herein, unless otherwise specified, refers to nutritional compositions in ready-to-drink liquid form, concentrated form, and nutritional liquids made by reconstituting the nutritional powders described herein prior to use.

The term “nutritional powder” as used herein, unless otherwise specified, refers to nutritional compositions in flowable or scoopable form that can be reconstituted with water or another aqueous liquid prior to consumption and includes both spraydried and drymixed/dryblended powders.

The term “nutritional semi-solid,” as used herein, unless otherwise specified, refers to nutritional compositions that are intermediate in properties, such as rigidity, between solids and liquids. Some semi-solids examples include puddings, gelatins, and doughs.

The term “nutritional semi-liquid,” as used herein, unless otherwise specified, refers to nutritional compositions that are intermediate in properties, such as flow properties, between liquids and solids. Some semi-liquids examples include thick shakes and liquid gels.

The terms “fat” and “oil” as used herein, unless otherwise specified, are used interchangeably to refer to lipid materials derived or processed from plants or animals. These terms also include synthetic lipid materials so long as such synthetic materials are suitable for oral administration to humans.

The term “shelf stable” as used herein, unless otherwise specified, refers to a nutritional product that remains commercially stable after being packaged and then stored at 18-24° C. for at least 3 months, including from about 6 months to about 24 months, and also including from about 12 months to about 18 months.

The term “individual” as used herein, refers generally to a preterm infant, infant, toddler, child, or adult.

The term “infant” as used herein, refers generally to individuals up to age 36 months of age, actual or corrected.

The term “preterm infant” as used herein, refers to those infants born at less than 37 weeks gestation, have a birth weight of less than 2500 gm, or both.

The term “infirmed adult” as used herein, refers to an adult in a nursing home, under hospital care, or an elderly adult (e.g., 60+ years of age).

The term “bovine immunoglobulin” as used herein refers to glycoprotein molecules, also referred to as antibodies, that are present in the milk of bovines, including IgA, IgD, IgE, IgG and IgM. In certain exemplary embodiments, bovine immunoglobulin refers to IgG present in bovine milk. In certain exemplary embodiments, bovine immunoglobulin refers to IgM present in bovine milk. In certain exemplary embodiments, the bovine immunoglobulin is a natural immunoglobulin.

The term “natural immunoglobulin” and “natural bovine immunoglobulin” are used interchangeably herein and refer to immunoglobulin that is non-specific and/or sourced from an animal that has not been stimulated or otherwise intentionally induced with antigens in a process intended to produce an increased response to human virus(es). In certain embodiments, the terms refer to immunoglobulin sourced from an animal that has not been hyperimmunized or otherwise intentionally exposed to conditions intended to increase activity against e.g., human virus (or the attendant induced inflammation). Those of ordinary skill in the art will recognize that cow herd vaccinations in certain jurisdictions include e.g., bovine viruses and other pathogens common to those species. The terms used herein refer to an animal that has not been stimulated to produce an increased response to a pathogen other than those specific to the particular species (e.g., cows immunized against bovine viruses).

As used herein, all concentrations expressed as either “μg/liter,” “mg/liter,” “mg/L,” “mcg/L,” “g/L,” etc., refer to ingredient concentrations within the described nutritional compositions as calculated on an as-fed basis (e.g., reconstituted for consumption in the case of nutritional powders), unless otherwise specified.

The term “reconstitute” or various other forms such as “reconstituted” or “reconstituting” all refer to the general act of adding a suitable amount of liquid, typically water, to a form of nutritional formulation that is not in its ready-to-drink liquid form, such as nutritional powder or a concentrated form of a nutritional liquid, thereby making the nutritional composition ready-to-drink.

The terms “susceptible” and “at risk” as used herein, unless otherwise specified, mean having little resistance to a certain condition or disease relative to the general population, including being genetically predisposed, having a family history of, and/or having symptoms of the condition or disease. The term refers to those having a vulnerability higher than the general population.

The terms “modulating” or “modulation” or “modulate” as used herein, unless otherwise specified, refer to the targeted movement of a selected characteristic. Those of ordinary skill in the art will understand that due to the complexity of the systems involved, in certain instances, modulating of a selected characteristic (e.g., a cytokine level) may involve appropriate sizing (i.e., right-sizing) of the cytokine to achieve the desired result, rather than merely reducing the level in all circumstances.

The term “ameliorate” as used herein, unless otherwise specified, means to eliminate, delay, or reduce the prevalence or severity of symptoms associated with a condition.

The term “an effective amount” is intended to qualify the amount of bovine immunoglobulin which will achieve the goal of decreasing the risk that the individual will suffer an adverse health event, including reducing one or more symptoms, while avoiding adverse side effects such as those typically associated with alternative therapies. The effective amount may be administered in one or more doses.

The terms “treating” and “treatment” as used herein, unless otherwise specified, includes delaying the onset of a condition, reducing the severity of symptoms of a condition, or eliminating some or all of the symptoms of a condition.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the general inventive concepts shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The bovine immunoglobulin can be formulated in suitable compositions and then, in accordance with the methods of the invention, administered to an individual in a form adapted to the chosen route of administration. The formulations include, but are not limited to, those suitable for oral or parenteral (including subcutaneous, intramuscular, intraperitoneal, intratumoral, and intravenous) administration. Oral administration, as defined herein, includes any form of administration in which the composition passes through the esophagus of the patient. For example, oral administration includes nasogastric intubation, in which a tube is run from through the nose to the stomach of the patient to administer food or drugs.

Oral formulations of the compositions disclosed herein include any solid, liquid, or powder formulation suitable for use herein, provided that such a formulation allows for the safe and effective oral delivery of the bovine immunoglobulin and optional nutritive components. In preferred embodiments, the oral formulation is a liquid nutritional composition or reconstitutable powder. Formulations of the present invention suitable for oral administration may be presented as discrete units such as tablets, troches, capsules, lozenges, wafers, or cachets, each containing a predetermined amount of the bovine immunoglobulin as a powder or granules or as a solution or suspension in an aqueous liquor or non-aqueous liquid such as a syrup, an elixir, an emulsion, or a draught.

The nutritional compositions and methods may comprise, consist of, or consist essentially of the essential elements of the compositions and methods as described herein, as well as any additional or optional element described herein or otherwise useful in oral or parenteral applications.

The general inventive concepts are based, in part, on the discovery that administration of bovine immunoglobulin can modulate, reduce, or blunt the cytokine cascade (often called a cytokine storm or unwanted cytokine burden) associated with certain infections, including viral infections. The reduction occurs independent of the immunoglobulin's capacity to bind to viral antigens. Further, the reduction can be achieved in the absence of altering basal cytokine levels in the individual. In certain embodiments, the cytokine cascade or storm may be triggered by the presence of a viral pathogen prior to infection of the first host cell and/or before the individual is symptomatic.

In certain exemplary embodiments, the general inventive concepts relate to a method of modulating respiratory virus-induced inflammation in an individual affected by a respiratory virus. In certain exemplary embodiments, the general inventive concepts relate to a method of reducing respiratory virus-induced inflammation in an individual affected by a respiratory virus. The method comprises administering a composition comprising from 0.01 g/L to 100 g/L of bovine immunoglobulin to the individual. The reduction is measured relative to infected individual who has not been administered the composition. Those of ordinary skill in the art will recognize that there are a variety of means for measuring the reduction of in inflammation, including measurement in a clinical setting. In certain embodiments, the individual affected by a respiratory virus is infected by the respiratory virus. In certain embodiments, the individual affected by a respiratory virus has measurable levels of virus but has not yet shown symptoms of infection. In certain exemplary embodiments, the method to decrease cytokine burden and ameliorate sickness/illness behavior symptoms by reducing respiratory virus-induced inflammation comprises administration of nutritional composition comprising from 0.01 g/L to 100 g/L of bovine immunoglobulin to the individual.

The cytokine cascade/storm is associated with and often leads to certain behavioral or physiological symptoms that detrimentally affect the individual and can hamper or prolong recovery. These behaviors/symptoms include e.g., impaired cognition, fever, anhedonia, loss of appetite, hyperalgesia, sleep disturbances, lethargy, chills, irritability, and skin hypersensitivity to touch, and present in infant/pediatric and adult populations. Modulating and/or controlling of the symptoms by reducing the cytokine burden to a level closer to basal may allow the body to more readily resolve the infection, while minimizing the undesirable symptoms. The general inventive concepts show that administration of bovine immunoglobulin reduces the magnitude of expression of pro-inflammatory cytokines associated with sickness behaviors including loss of appetite. By replacing a portion of a protein source with increased concentrations of natural bovine immunoglobulins, an individual will experience a modulation/right-sizing/reduction in the magnitude of expression of certain virus-induced cytokines and a corresponding reduction in infection-related inflammation and/or morbidity. This discovery is important because it differs from drug/pharmaceutical interventions as it is preventative and avoids unwanted tampering with basal cytokine levels. Thus, the general inventive concepts differ from conventional attempts to introducing non-specific immunoglobulins, which attempt to reduce viral load and disease severity through direct interactions with the virus or though modulation of epithelial cell glycosylation patterns.

Bovine Immunoglobulin

The compositions according to the general inventive concepts include a source of bovine immunoglobulin. In certain exemplary embodiments, the bovine immunoglobulin included in the compositions is natural bovine immunoglobulin. In certain exemplary embodiments, the bovine immunoglobulin is present in the composition in an amount of 0.01 g/L to 100 g/L. In certain exemplary embodiments, the bovine immunoglobulin is present in the composition in an amount of 0.01 g/L to 50 g/L. In certain exemplary embodiments, the bovine immunoglobulin is present in the composition in an amount of 0.01 g/L to 25 g/L. In certain exemplary embodiments, the bovine immunoglobulin is present in the composition in an amount of 0.01 g/L to 20 g/L. In certain exemplary embodiments, the bovine immunoglobulin is present in the composition in an amount of 0.01 g/L to 15 g/L. In certain exemplary embodiments, the bovine immunoglobulin is present in the composition in an amount of 0.01 g/L to 10 g/L. In certain exemplary embodiments, the bovine immunoglobulin is present in the composition in an amount of 0.01 g/L to 9 g/L. In certain exemplary embodiments, the bovine immunoglobulin is present in the composition in an amount of 0.01 g/L to 8 g/L. In certain exemplary embodiments, the bovine immunoglobulin is present in the composition in an amount of 0.01 g/L to 7 g/L. In certain exemplary embodiments, the bovine immunoglobulin is present in the composition in an amount of 0.01 g/L to 6 g/L. In certain exemplary embodiments, the bovine immunoglobulin is present in the composition in an amount of 0.01 g/L to 5 g/L. In certain exemplary embodiments, the bovine immunoglobulin is present in the composition in an amount of 0.01 g/L to 4 g/L. In certain exemplary embodiments, the bovine immunoglobulin is present in the composition in an amount of 0.01 g/L to 3 g/L. In certain exemplary embodiments, the bovine immunoglobulin is present in the composition in an amount of 0.01 g/L to 2 g/L. In certain exemplary embodiments, the bovine immunoglobulin is present in the composition in an amount of 0.01 g/L to 1 g/L.

In certain exemplary embodiments, the bovine immunoglobulin is present in the composition in an amount of 0.1 g/L to 1 g/L. In certain exemplary embodiments, the bovine immunoglobulin is present in the composition in an amount of 0.2 g/L to 1 g/L. In certain exemplary embodiments, the bovine immunoglobulin is present in the composition in an amount of 0.3 g/L to 1 g/L. In certain exemplary embodiments, the bovine immunoglobulin is present in the composition in an amount of 0.4 g/L to 1 g/L. In certain exemplary embodiments, the bovine immunoglobulin is present in the composition in an amount of 0.5 g/L to 1 g/L. In certain exemplary embodiments, the bovine immunoglobulin is present in the composition in an amount of 0.6 g/L to 1 g/L. In certain exemplary embodiments, the bovine immunoglobulin is present in the composition in an amount of 0.7 g/L to 1 g/L. In certain exemplary embodiments, the bovine immunoglobulin is present in the composition in an amount of 0.8 g/L to 1 g/L. In certain exemplary embodiments, the bovine immunoglobulin is present in the composition in an amount of 0.9 g/L to 1 g/L.

In certain exemplary embodiments, the bovine immunoglobulin is present in the composition in an amount of 0.5 g/L to 10 g/L. In certain exemplary embodiments, the bovine immunoglobulin is present in the composition in an amount of 1 g/L to 10 g/L. In certain exemplary embodiments, the bovine immunoglobulin is present in the composition in an amount of 2 g/L to 10 g/L. In certain exemplary embodiments, the bovine immunoglobulin is present in the composition in an amount of 3 g/L to 10 g/L. In certain exemplary embodiments, the bovine immunoglobulin is present in the composition in an amount of 4 g/L to 10 g/L. In certain exemplary embodiments, the bovine immunoglobulin is present in the composition in an amount of 5 g/L to 10 g/L. In certain exemplary embodiments, the bovine immunoglobulin is present in the composition in an amount of 6 g/L to 10 g/L. In certain exemplary embodiments, the bovine immunoglobulin is present in the composition in an amount of 7 g/L to 10 g/L. In certain exemplary embodiments, the bovine immunoglobulin is present in the composition in an amount of 8 g/L to 10 g/L. In certain exemplary embodiments, the bovine immunoglobulin is present in the composition in an amount of 9 g/L to 10 g/L. In certain exemplary embodiments, the bovine immunoglobulin may comprise a portion of the protein that would otherwise make up a nutritional composition, including but not limited to a majority of the protein in the nutritional composition.

While the general inventive concepts discussed herein refer to bovine immunoglobulin, in addition to milk from a bovine source, in certain exemplary embodiments, the general inventive concepts contemplate immunoglobulin sourced from other mammals including, but not limited to immunoglobulin sourced from one or more of water buffalo, goat, sheep, camel, donkey, horse, reindeer and yak.

Compositions containing bovine immunoglobulin, as defined herein, can be used to ameliorate or treat the conditions or diseases described herein. The general inventive concepts and examples described herein show that while not all respiratory viruses respond directly to immunoglobulin treatment with respect to reducing viral load in epithelial cell cultures, immunoglobulin treatment does appear to reduce the overall burden of key cytokines involved in fever and various aspects of sickness/illness behaviors including loss of appetite and anhedonia.

### Long Chain Polyunsaturated Fatty Acids

In addition to the bovine immunoglobulin described above, the nutritional compositions according to the general inventive concepts may include Long Chain Polyunsaturated Fatty Acids (LCPUFAs). LCPUFAs are included in the nutritional compositions to provide nutritional support, as well as to modulate or treat the conditions or diseases described herein.

Exemplary LCPUFAs for use in the nutritional compositions include, for example, ω-3 LCPUFAs and ω-6 LCPUFAs. Specific LCPUFAs include docosahexaenoic acid (DHA), eicosapentaenoic acid (EPA), docosapentaenoic acid (DPA), arachidonic acid (ARA), linoleic acid, linolenic acid (alpha linolenic acid) and gamma-linolenic acid derived from oil sources such as plant oils, marine plankton, fungal oils, krill oil and fish oils. In one particular aspect, the LCPUFAs are derived from fish oils such as menhaden, salmon, anchovy, cod, halibut, tuna, or herring oil. Particularly preferred LCPUFAs for use in the nutritional compositions include DHA, ARA, EPA, DPA, and combinations thereof.

In order to reduce potential side effects of high dosages of LCPUFAs including DHA, ARA, EPA, DPA, in the nutritional compositions, the content of DHA, ARA, EPA, DPA, preferably does not exceed 5% by weight of the total fat content, including below 2% by weight of the total fat content, and including below 1% by weight of the total fat content in the nutritional composition.

The LCPUFA may be provided as free fatty acids, in triglyceride form, in diglyceride form, in monoglyceride form, in phospholipid form, in esterified form or as a mixture of one or more of the above, preferably in triglyceride form.

The nutritional compositions as described herein will typically comprise total concentrations of ARA, DHA, EPA, and DPA of from about 0.001 g/L to about 1 g/L, including from about 0.01 g/L to about 1 g/L, and about 0.1 g/L to about 1 g/L.

In an exemplary embodiment, the nutritional compositions include a long chain polyunsaturated fatty acid component comprising DHA and ARA in a concentration of from about 0.17 mg/mL to about 0.33 mg/mL, including from about 0.17 mg/mL to about 0.26 mg/mL of ARA and DHA. In an exemplary embodiment, the nutritional compositions include DHA in a concentration of from about 0.025 mg/mL to about 0.130 mg/mL. In another embodiment, the nutritional compositions include ARA in a concentration of from about 0.080 mg/mL to about 0.350 mg/mL. In yet another embodiment, the nutritional compositions include combinations of DHA and ARA such that the ratio of DHA to ARA ranges from about 1:4 to about 1:2.

### Antioxidants

Additionally, the nutritional compositions may comprise one or more antioxidants in combination with the bovine immunoglobulin (and optionally LCPUFAs and/or nucleotides) to provide nutritional support, as well as to provide a synergistic benefit to the end user, such as a synergistic benefit in modulating or treating one or more of the conditions or diseases described herein.

Any antioxidants suitable for oral administration may be included for use in the nutritional compositions according to the general inventive concepts, including, for example, vitamin A, vitamin E, vitamin C, retinol, tocopherol, and carotenoids, including lutein, beta-carotene, zeaxanthin, and lycopene, and combinations thereof, for example.

As noted, the antioxidants for use in the nutritional compositions may be used with the bovine immunoglobulin alone or in combination with LCPUFAs, and/or nucleotides.

It is generally preferable that the nutritional compositions comprise at least one carotenoid selected from lutein, lycopene, zeaxanthin, and beta-carotene to provide a total amount of carotenoid of from about 0.001 μg/mL to about 10 μg/mL. More particularly, the nutritional compositions comprise lutein in an amount of from about 0.001 μg/mL to about 10 μg/mL, including from about 0.001 μg/mL to about 5 μg/mL, including from about 0.001 μg/mL to about 0.0190 μg/mL, including from about 0.001 μg/mL to about 0.0140 μg/mL, and also including from about 0.044 μg/mL to about 5 μg/mL of lutein. It is also generally preferable that the nutritional compositions comprise from about 0.001 μg/mL to about 10 μg/mL, including from about 0.001 μg/mL to about 5 μg/mL, from about 0.001 μg/mL to about 0.0130 μg/mL, including from about 0.001 μg/mL to about 0.0075 μg/mL, and also including from about 0.0185 μg/mL to about 5 μg/mL of lycopene. It is also generally preferable that the nutritional compositions comprise from about 1 μg/mL to about 10 μg/mL, including from about 1 μg/mL to about 5 μg/mL, including from about 0.001 μg/mL to about 0.025 μg/mL, including from about 0.001 μg/mL to about 0.011 μg/mL, and also including from about 0.034 μg/mL to about 5 μg/mL of beta-carotene. It should be understood that any combination of these amounts of beta-carotene, lutein, zeaxanthin, and lycopene can be included in the nutritional compositions according to the general inventive concepts. Other carotenoids may optionally be included in the nutritional compositions as described herein. Any one or all of the carotenoids included in the nutritional compositions described herein may be from a natural source or artificially synthesized.

Each of the carotenoids in the selected combinations can be obtained from any known or otherwise suitable material source for use in nutritional compositions, and each can be provided individually, or all together, or in any combination and from any number of sources, including sources such as multivitamin premixes containing other vitamins or minerals in combination with one or more of the carotenoids as described herein. Non-limiting examples of some suitable sources of lutein, lycopene, beta-carotene, or combinations thereof include LycoVit® lycopene (available from BASF, Mount Olive, N.J.), Lyc-O-Mato® tomato extract in oil, powder, or bead form (available from LycoRed Corp., Orange, N.J.), beta-carotene, lutein, or lycopene (available from DSM Nutritional Products, Parsippany, N.J.), FloraGLO® lutein (available from Kemin Health, Des Moines, Iowa), Xangold® Natural Lutein Esters (available from Cognis, Cincinnati, Ohio), and Lucarotin® beta-carotene (available from BASF, Mount Olive, N.J.).

### Nucleotides

In addition to the bovine immunoglobulin, the nutritional compositions according to the general inventive concepts may additionally comprise nucleotides and/or nucleotide precursors selected from the group consisting of nucleosides, purine bases, pyrimidine bases, ribose and deoxyribose. The nucleotide may be in monophosphate, diphosphate, or triphosphate form. The nucleotide may be a ribonucleotide or a deoxyribonucleotide. The nucleotides may be monomeric, dimeric, or polymeric (including RNA and DNA). The nucleotide may be present in the nutritional composition as a free acid or in the form of a salt, preferably a monosodium salt.

Suitable nucleotides and/or nucleosides for use in the nutritional compositions include one or more of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, and inosine 5′-monophosphate.

In an exemplary embodiment, the nucleotides are present in the nutritional compositions in a total amount of about 72 mg/L to about 300 mg/L of the ready to feed form of the nutritional composition. In an exemplary embodiment, the nucleotides are present in the nutritional composition in a total amount of about 72 mg/L of the nutritional product and comprise about 43% cytidine 5′-monophosphate, about 18.5% uridine 5′-monophosphate, about 16.5% adenosine 5′-monophosphate and about 22% guanosine 5′-monophosphate by total weight of nucleotides.

In an exemplary embodiment, the nucleotides are present in the nutritional products in a total amount of about 72 mg/L of the nutritional product and comprise about 29 to 39 mg of cytidine 5′-monophosphate; 15 to 21 mg of uridine 5′-monophosphate; 10 to 15 mg of adenosine 5′-monophosphate; and 14 to 20 mg of guanosine 5′-monophosphate.

In an exemplary embodiment, the nucleotides are present in the weight ratio of cytidine 5′-monophosphate: uridine 5′-monophosphate is from about 1.5:1 to about 2.6:1; of cytidine 5′-monophosphate: adenosine 5′-monophosphate is from about 2:1 to about 3.9:1; and of cytidine 5′-monophosphate: guanosine 5′-monophosphate is from about 1.75:1 to about 2.8:1.

As noted, the nucleotides for use in the nutritional compositions may be used with the bovine immunoglobulin alone or in combination with LCPUFAs, and/or antioxidants. In certain embodiments, the nutritional composition includes a combination of the bovine immunoglobulin and one or more of LCPUFAs, antioxidants, and nucleotides as described herein such that the composition provides a synergistic benefit to the end user, such as a synergistic benefit in modulating or treating one or more of the aforementioned conditions or diseases.

In addition to the specific functional ingredients described herein, the nutritional composition includes one or more ingredients that help satisfy the individual's nutritional requirements. The optional nutrients can provide up to about 1000 kcal of energy per serving or dose, including from about 25 to about 900 kcal, from about 75 to about 700 kcal, from about 150 to about 500 kcal, from about 350 to about 500 kcal, or from about 200 to about 300 kcal.

The nutritional compositions may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit as described below.

The nutritional compositions including the bovine immunoglobulin may be formulated to include at least one of protein, fat, and carbohydrate. In many embodiments, the nutritional compositions will include the bovine immunoglobulin with protein, carbohydrate and fat. Although total concentrations or amounts of the fat, protein, and carbohydrates may vary depending upon the product type (i.e., nutritional formula), product form (i.e., nutritional solid, powder, ready-to-feed liquid, or concentrated liquid) and targeted dietary needs of the intended user, such concentrations or amounts most typically fall within one of the following embodied ranges, inclusive of any other essential fat, protein, and/or carbohydrate ingredients as described herein.

### Nutritional Compositions

Nutritional compositions, as discussed herein, include at least one of fat, carbohydrate, and protein in addition to the bovine immunoglobulin.

Where present, carbohydrate concentrations most typically will range from about 5% to about 40%, including from about 7% to about 30%, including from about 10% to about 25%, by weight of the nutritional composition. Where present, fat concentrations most typically range from about 1% to about 30%, including from about 2% to about 15%, and also including from about 3% to about 10%, by weight of the nutritional composition. Where present, protein concentrations most typically range from about 0.5% to about 30%, including from about 1% to about 15%, and also including from about 2% to about 10%, by weight of the nutritional composition.

The amount of any or all of the carbohydrates, fats, and proteins in any of the nutritional compositions (e.g., infant formula, pediatric formula, adult formula) described herein may also be characterized as a percentage of total calories in the nutritional composition as set forth in the following table. These macronutrients for nutritional compositions according to the general inventive concepts are most typically formulated within any of the caloric ranges (embodiments A-F) described in the following table (each numerical value is preceded by the term “about”).

TABLE 1
Exemplary macronutrient profiles of nutritional compositions

| | Embodiment A | Embodiment B | Embodiment C |
|---|---|---|---|
| Nutrient | (% Total Cal.) | (% Total Cal.) | (% Total Cal.) |
| Carbohydrate | 0-98 | 2-96 | 10-75 |
| Protein | 0-98 | 2-96 | 5-70 |
| Fat | 0-98 | 2-96 | 20-85 |
| | Embodiment D | Embodiment E | Embodiment F |
| | (% Total Cal.) | (% Total Cal.) | (% Total Cal.) |
| Carbohydrate | 30-50 | 25-50 | 25-50 |
| Protein | 15-35 | 10-30 | 5-30 |
| Fat | 35-55 | 1-20 | 2-20 |

### Fat

The nutritional compositions according to the general inventive concepts may, in addition to the LCPUFAs, comprise an additional source or sources of fat. Suitable additional sources of fat for use herein include any fat or fat source that is suitable for use in an oral nutritional product and is compatible with the essential elements and features of such products. Most typically the fat may be an emulsified fat, concentrations of which may range from about 1% to about 30%, including from about 2% to about 15%, and also including from about 4% to about 10%, by weight. In an exemplary embodiment, the additional fat is derived from short chain fatty acids.

Additional non-limiting examples of suitable fats or sources thereof for use in the nutritional products described herein include coconut oil, fractionated coconut oil, soybean oil, corn oil, olive oil, safflower oil, high oleic safflower oil, oleic acids (EMERSOL 6313 OLEIC ACID, Cognis Oleochemicals, Malaysia), MCT oil (medium chain triglycerides), sunflower oil, high oleic sunflower oil, palm and palm kernel oils, palm olein, canola oil, marine oils, fish oils, fungal oils, algae oils, cottonseed oils, and combinations thereof. Lipid sources of arachidonic acid and docosahexaenoic acid include, but are not limited to, marine oil, egg yolk oil, and fungal or algal oil.

Numerous commercial sources for these fats are readily available and known to one practicing the art. For example, soy and canola oils are available from Archer Daniels Midland of Decatur, Ill. Corn, coconut, palm and palm kernel oils are available from Premier Edible Oils Corporation of Portland, Organ. Fractionated coconut oil is available from Henkel Corporation of LaGrange, Ill. High oleic safflower and high oleic sunflower oils are available from SVO Specialty Products of Eastlake, Ohio. Marine oil is available from Mochida International of Tokyo, Japan. Olive oil is available from Anglia Oils of North Humberside, United Kingdom. Sunflower and cottonseed oils are available from Cargil of Minneapolis, Minn. Safflower oil is available from California Oils Corporation of Richmond, Calif.

In addition to these food grade oils, structured lipids may be incorporated into the food product if desired. Structured lipids are known in the art. A concise description of structured lipids can be found in INFORM, Vol. 8, No. 10, page 1004; entitled Structured lipids allow fat tailoring (October 1997). Also see U.S. Pat. No. 4,871,768. Structured lipids are predominantly triacylglycerols containing mixtures of medium and long chain fatty acids on the same glycerol nucleus. Structured lipids and their use in enteral formula are also described in U.S. Pat. Nos. 6,194,379 and 6,160,007.

Optionally, ω-3 fatty acids may comprise up to approximately 5% of the oil blend, preferably the ω-3 fatty acids largely consist of the longer chain forms, eicosapentaenoic acid (EPA) and docosahexaenoic acid (DHA). Dietary oils used in the preparation of the nutritional composition generally contain ω-3 fatty acids in the triglyceride form and include, but are not limited to canola, medium chain triglycerides, fish, soybean, soy lecithin, corn, safflower, sunflower, high-oleic sunflower, high-oleic safflower, olive, borage, black currant, evening primrose and flaxseed oil. Optionally, the weight ratio of ω-6 fatty acids to ω-3 fatty acids in the lipid blend according to the invention is about 0.1 to 3.0. The daily delivery of ω-3 fatty acids should be at least 450 mg and may vary depending on body weight, sex, age and medical condition of the individual. As mentioned, higher levels are desired for adult human consumption: for example, from about 0.5 to 50 gm daily, more preferably from about 2.5 to 5 gm daily

### Protein

In certain exemplary embodiments, the nutritional compositions according to the general inventive concepts include protein in addition to the bovine immunoglobulin. In certain exemplary embodiments, the bovine immunoglobulin replaces a portion of the protein that otherwise would be present in the nutritional composition, including, up to a majority of the protein component. Any protein source that is suitable for use in oral nutritional compositions and is compatible with the essential elements and features of the general inventive concepts may be used.

Non-limiting examples of suitable proteins or sources thereof for use in the nutritional compositions include hydrolyzed, partially hydrolyzed or non-hydrolyzed proteins or protein sources, which may be derived from any known or otherwise suitable source such as milk (e.g., casein, whey), animal (e.g., meat, fish), cereal (e.g., rice, corn), vegetable (e.g., soy) or combinations thereof. Non-limiting examples of such proteins include milk protein isolates, milk protein concentrates as described herein, casein protein isolates, extensively hydrolyzed casein, whey protein, sodium or calcium caseinates, whole cow milk, partially or completely defatted milk, soy protein isolates, and soy protein concentrates.

In an exemplary embodiment, the protein source is a hydrolyzed protein, i.e., a protein hydrolysate. In this context, the terms “hydrolyzed protein” or “protein hydrolysates” are used interchangeably herein and include extensively hydrolyzed proteins, wherein the degree of hydrolysis is most often at least about 20%, including from about 20% to about 80%, and also including from about 30% to about 80%, even more preferably from about 40% to about 60%. The degree of hydrolysis is the extent to which peptide bonds are broken by a hydrolysis method. The degree of protein hydrolysis for purposes of characterizing the extensively hydrolyzed protein component of these embodiments is easily determined by one of ordinary skill in the formulation arts by quantifying the amino nitrogen to total nitrogen ratio (AN/TN) of the protein component of the selected liquid formulation. The amino nitrogen component is quantified by USP titration methods for determining amino nitrogen content, while the total nitrogen component is determined by the Tecator Kjeldahl method, all of which are well known methods to one of ordinary skill in the analytical chemistry art.

Suitable hydrolyzed proteins include soy protein hydrolysate, casein protein hydrolysate, whey protein hydrolysate, rice protein hydrolysate, potato protein hydrolysate, fish protein hydrolysate, egg albumen hydrolysate, gelatin protein hydrolysate, combinations of animal and vegetable protein hydrolysates, and combinations thereof. Particularly preferred protein hydrolysates include whey protein hydrolysate and hydrolyzed sodium caseinate.

When used in the nutritional compositions, the protein source may include at least about 20% (by weight total protein) protein hydrolysate, including from about 30% to 100% (by weight total protein) protein hydrolysate, and including from about 40% to about 80% (by weight total protein) protein hydrolysate, and including about 50% (by weight total protein) protein hydrolysate.

### Carbohydrate

In an exemplary embodiment, the nutritional compositions according to the general inventive concepts include carbohydrates that are suitable for use in an oral nutritional composition and are compatible with the essential elements and features of the general inventive concepts.

Non-limiting examples of suitable carbohydrates or sources thereof for use in the nutritional compositions described herein include maltodextrin, hydrolyzed or modified starch or cornstarch, glucose polymers, corn syrup, corn syrup solids, rice-derived carbohydrates, pea-derived carbohydrates, potato-derived carbohydrates, tapioca, sucrose, glucose, fructose, lactose, high fructose corn syrup, honey, sugar alcohols (e.g., maltitol, erythritol, sorbitol), artificial sweeteners (e.g., sucralose, acesulfame potassium, stevia) and combinations thereof. A particularly desirable carbohydrate is a low dextrose equivalent (DE) maltodextrin.

### Optional Ingredients

The nutritional compositions according to the general inventive concepts may further comprise other optional components that may modify the physical, chemical, aesthetic or processing characteristics of the products or serve as pharmaceutical or additional nutritional components when used in the targeted population. Many such optional ingredients are known or otherwise suitable for use in medical food or other nutritional products or pharmaceutical dosage forms and may also be used in the compositions herein, provided that such optional ingredients are safe for oral administration and are compatible with the essential and other ingredients in the selected product form.

Non-limiting examples of such optional ingredients include preservatives, emulsifying agents, buffers, fructooligosaccharides, fiber, galactooligosaccharides, polydextrose, and other prebiotics, probiotics, pharmaceutical actives, anti-inflammatory agents, additional nutrients as described herein, colorants, flavors, thickening agents and stabilizers, emulsifying agents, lubricants, and so forth.

The nutritional compositions may further comprise a sweetening agent, preferably including at least one sugar alcohol such as maltitol, erythritol, sorbitol, xylitol, mannitol, isolmalt, and lactitol, and also preferably including at least one artificial or high potency sweetener such as acesulfame K, aspartame, sucralose, saccharin, stevia, and tagatose. These sweetening agents, especially as a combination of a sugar alcohol and an artificial sweetener, are especially useful in formulating liquid beverage embodiments according to the general inventive concepts having a desirable favor profile. These sweetener combinations are especially effective in masking undesirable flavors sometimes associated with the addition of vegetable proteins to a liquid beverage. Optional sugar alcohol concentrations in the nutritional product may range from at least 0.01%, including from about 0.1% to about 10%, and also including from about 1% to about 6%, by weight of the nutritional product. Optional artificial sweetener concentrations may range from about 0.01%, including from about 0.05% to about 5%, also including from about 0.1% to about 1.0%, by weight of the nutritional product.

The nutritional compositions may further comprise a flowing agent or anti-caking agent to retard clumping or caking of the powder over time and to make a powder embodiment flow easily from its container. Any known flowing or anti-caking agents that are known or otherwise suitable for use in a nutritional powder or product form are suitable for use herein, non-limiting examples of which include tricalcium phosphate, silicates, and combinations thereof. The concentration of the flowing agent or anti-caking agent in the nutritional composition varies depending upon the product form, the other selected ingredients, the desired flow properties, and so forth, but most typically range from about 0.1% to about 4%, including from about 0.5% to about 2%, by weight of the nutritional composition.

The nutritional compositions may further comprise a stabilizer. Any stabilizer that is known or otherwise suitable for use in a nutritional composition is also suitable for use herein, some non-limiting examples of which include gums such as xanthan gum. The stabilizer may represent from about 0.1% to about 5.0%, including from about 0.5% to about 3%, including from about 0.7% to about 1.5%, by weight of the nutritional composition.

The nutritional compositions may further comprise any of a variety of other vitamins or related nutrients, non-limiting examples of which include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and combinations thereof. The food products preferably include, but are not limited to, the following vitamins and minerals: calcium, phosphorus, sodium, chloride, magnesium, manganese, iron, copper, zinc, selenium, iodine, chromium, molybdenum, conditionally essential nutrients m-inositol, carnitine and taurine, and Vitamins A, C, D, E (including natural vitamin E), K and the B complex, and mixtures thereof.

The nutritional compositions may further comprise any of a variety of other additional minerals, non-limiting examples of which include calcium, phosphorus, magnesium, iron, zinc, manganese, copper, sodium, potassium, molybdenum, chromium, chloride, and combinations thereof.

The nutritional compositions also may contain fiber and fiber-like stabilizers. Suitable sources of fiber and/or stabilizers include, but are not limited to, xanthan gum, guar gum, gum arabic, gum ghatti, gum karaya, gum tracacanth, agar, furcellaran, gellan gum, locust bean gum, pectin, low and high methoxy pectin, oat and barley glucans, carrageenans, psyllium, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, FOS (fructooligosaccharides), and mixtures thereof. Numerous commercial sources of soluble dietary fibers are available. For example, gum arabic, hydrolyzed carboxymethylcellulose, guar gum, pectin and the low and high methoxy pectins are available from TIC Gums, Inc. of Belcamp, Md. The oat and barley glucans are available from Mountain Lake Specialty Ingredients, Inc. of Omaha, Nebr. Psyllium is available from the Meer Corporation of North Bergen, N.J. while the carrageenan is available from FMC Corporation of Philadelphia, Pa.

In addition to fiber, the nutritional compositions may also contain oligosaccharides such as fructooligosaccharides (FOS) or galactooligosaccharides (GOS). Oligosaccharides are rapidly and extensively fermented to short chain fatty acids by anaerobic microorganisms that inhabit the large bowel. These oligosaccharides are preferential energy sources for most Bifidobacterium species, but are not utilized by potentially pathogenic organisms such as Clostridium perfringens, C. difficile, or Escherichia coli.

The nutritional products may additionally comprise one or more thickeners (i.e., thickening agents). The addition of thickeners reduces the incidences of paresthesia by inducing the feeling of satiety, which prolongs gastric transit time as discussed above.

### Product Forms

The compositions according to the general inventive concepts may be formulated and administered in any known or otherwise suitable oral product form. Any solid, liquid, semi-solid, and semi-liquid, or powder product form, including combinations or variations thereof, are suitable for use herein, provided that such forms allow for safe and effective oral delivery to the individual of the essential ingredients as also defined herein.

The compositions may be in any product form comprising the ingredients described herein, and which is safe and effective for oral administration. The compositions may be formulated to include only the ingredients described herein, or may be modified with optional ingredients to form a number of different product forms.

The compositions according to the general inventive concepts are desirably formulated as dietary product forms, which are defined herein as those embodiments comprising the ingredients according to the general inventive concepts in a product form that then contains at least one of fat, protein, and carbohydrate, and preferably also contains vitamins, minerals, or combinations thereof. The nutritional compositions will comprise at least bovine immunoglobulin, desirably in combination with at least one of protein, fat, vitamins, and minerals, to produce a nutritional composition.

Liquid compositions include both concentrated and ready-to-feed liquids. These nutritional liquids are most typically formulated as suspensions or emulsions, although other liquid forms are within the scope of the general inventive concepts. Nutritional compositions in the form of emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional emulsions may be and typically are shelf stable. The nutritional emulsions typically contain up to about 95% by weight of water, including from about 50% to about 95%, also including from about 60% to about 90%, and also including from about 70% to about 85%, of water by weight of the nutritional emulsions. The nutritional emulsions may have a variety of product densities, but most typically have a density greater than about 1.03 g/mL, including greater than about 1.04 g/mL, including greater than about 1.055 g/mL, including from about 1.06 g/mL to about 1.12 g/mL, and also including from about 1.085 g/mL to about 1.10 g/mL.

The nutritional emulsions may have a caloric density tailored to the nutritional needs of the ultimate user, although in most instances the emulsions comprise generally at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the emulsion may have a caloric density of from about 50-100 kcal/liter to about 660 kcal/liter, including from about 150 kcal/liter to about 500 kcal/liter. In an exemplary embodiment, the emulsion may have a caloric density of 25, or 50, or 75, or 100 kcal/liter.

The nutritional emulsion may have a pH ranging from about 2.5 to about 8, but are most advantageously in a range of from about 4.5 to about 7.5, including from about 5.5 to about 7.3, including from about 6.2 to about 7.2.

Although the serving size for the nutritional emulsion can vary depending upon a number of variables, a typical serving size is generally at least about 1 mL, or even at least about 2 mL, or even at least about 5 mL, or even at least about 10 mL, or even at least about 25 mL, including ranges from about 1 mL to about 475 mL, including from about 30 mL to about 360 mL, including from about 60 mL to about 300 mL, and including from about 60 mL to about 240 mL.

The compositions in the form of solids may be in any solid form but are typically in the form of flowable or substantially flowable particulate compositions, or at least particulate compositions. Particularly suitable nutritional solid product forms include spray dried, agglomerated and/or dry blended powder compositions. The compositions can easily be scooped and measured with a spoon or similar other device, and can easily be reconstituted by the intended user with a suitable aqueous liquid, typically water, to form a nutritional composition for immediate oral or enteral use. In this context, “immediate” use generally means within about 48 hours, most typically within about 24 hours, preferably right after reconstitution.

The nutritional powders may be reconstituted with water prior to use to a caloric density tailored to the nutritional needs of the ultimate user, although in most instances the powders are reconstituted with water to form compositions comprising at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the reconstituted powder may have a caloric density of from about 50-100 kcal/liter to about 660 kcal/liter, including from about 150 kcal/liter to about 500 kcal/liter. In an exemplary embodiment, the emulsion may have a caloric density of 25, or 50, or 75, or 100 kcal/liter.

### Methods of Manufacture

The nutritional compositions according to the general inventive concepts may be prepared by any known or otherwise effective manufacturing technique for preparing the nutritional compositions. Many such techniques are known for any given product form such as nutritional liquids or powders and can easily be applied by one of ordinary skill in the art to the nutritional compositions described herein.

The nutritional compositions according to the general inventive concepts can therefore be prepared by any of a variety of known or otherwise effective formulation or manufacturing methods. In one suitable manufacturing process, for example, at least three separate slurries are prepared, including a protein-in-fat (PIF) slurry, a carbohydrate-mineral (CHO-MIN) slurry, and a protein-in-water (PIW) slurry. The PIF slurry is formed by heating and mixing the oil (e.g., canola oil, corn oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra-trace minerals (TM/UTM premix), thickening or suspending agents (e.g., avicel, gellan, and carrageenan). The resulting CHO-MIN slurry is held for 10 minutes with continued heat and agitation before adding additional minerals (e.g., potassium chloride, magnesium carbonate, potassium iodide, etc.), and/or carbohydrates (e.g., fructooligosaccharide, sucrose, corn syrup, etc.). The PIW slurry is then formed by mixing with heat and agitation the remaining protein, if any.

The resulting slurries are then blended together with heated agitation and the pH adjusted to 6.6-7.0, after which the composition is subjected to high-temperature short-time (HTST) processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level. This emulsion can then be further diluted, heat-treated, and packaged to form a ready-to-feed or concentrated liquid, or it can be heat-treated and subsequently processed and packaged as a reconstitutable powder, e.g., spray dried, drymixed, agglomerated.

The nutritional solid, such as a spray dried nutritional powder or drymixed nutritional powder, may be prepared by any collection of known or otherwise effective techniques, suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising predigested fat, and optionally protein, carbohydrate, and other sources of fat as described above, and then spray drying the slurry or liquid to produce a spray dried nutritional powder. The method may further comprise the step of spray drying, drymixing, or otherwise adding additional nutritional ingredients, including any one or more of the ingredients described herein, to the spray dried nutritional powder.

Other suitable methods for making nutritional compositions are described, for example, in U.S. Pat. No. 6,365,218 (Borschel, et al.), U.S. Pat. No. 6,589,576 (Borschel, et al.), U.S. Pat. No. 6,306,908 (Carlson, et al.), U.S. Patent Application No. 20030118703 A1 (Nguyen, et al.), which descriptions are incorporated herein by reference to the extent that they are consistent herewith.

### Methods of Use

The methods of use according to the general inventive concepts include the oral administration of the compositions that include bovine immunoglobulin alone, or in combination with one or more functional ingredients including PUFAs, nucleotides, and carotenoids to improve or modulate at least one of the conditions or diseases discussed herein.

The compositions as described herein can be administered to individuals including infants, pediatric individuals, and adults (including elderly or infirm adults) generally, or may, in certain embodiments, be administered to a specific subclass of individuals that are “in need thereof,” that is, to specific individuals that would particularly benefit by administration of the compositions. For example, a specific individual may be “in need of” the composition as described herein if they are susceptible to (i.e., have one or more of a genetic predisposition, a family history of, and symptoms of the disease or condition) diseases and conditions that can impair/reduce function in one or more of the areas discussed herein.

The individual desirably consumes at least one serving of the composition daily, and in some embodiments, may consume two, three, or even more servings per day. Each serving is desirably administered as a single, undivided dose, although the serving may also be divided into two or more partial or divided servings to be taken at two or more times during the day. The methods according to the general inventive concepts include continuous day after day administration, as well as periodic or limited administration, although continuous day after day administration is generally desirable. The methods according to the general inventive concepts are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least about 18-24 months, desirably as a long term, continuous, daily, dietary source or supplement.

Based on the foregoing, because some of the method embodiments according to the general inventive concepts are directed to specific subsets or subclasses of identified individuals (that is, the subset or subclass of individuals “in need” of assistance in addressing one or more specific diseases or specific conditions noted herein), not all individuals will fall within the subset or subclass of individuals as described herein for certain diseases or conditions. For example, in certain instances not all infirm adults will specifically fall within the class. Rather, it is individuals which would be considered to have an increased susceptibility over the general class of individuals sue to one or more of the risk factors described above.

Furthermore, sickness/illness behaviors are potently associated with increases in proinflammatory cytokine concentrations. Thus, the general inventive concepts contemplate that immunoglobulin-induced modulation in these cytokines or cytokine concentrations could synergize with nutrients such as vitamin B-6 and choline to improve the positive affects described herein. Consumption of functional ingredients and essential micronutrients such as zinc have been shown to decrease respiratory virus load and disease severity. By inclusion in nutritional compositions containing vitamin B-6, mood (positive affect) should also be improved. Currently, consumers lack effective options for mitigating this sickness/illness-related loss of appetite and anhedonia.

### Examples

A nutritional composition comprising a bovine immunoglobulin alone or in combination with one or more of LCPUFAs, antioxidants, and nucleotides is administered to an individual. The nutritional composition modulates (and in certain embodiments, reduces) the cytokine burden associated with a respiratory viral infection. Inflammation is thereby reduced which leads to amelioration and/or treating of one or more conditions selected from impaired cognition, fever, anhedonia, loss of appetite, hyperalgesia, sleep disturbances, lethargy, chills, irritability, and skin hypersensitivity to touch.

The following examples illustrate exemplary embodiments and/or features of the methods and nutritional compositions according to the general inventive concepts. The examples are given solely for the purpose of illustration and are not to be construed as limitations of the general inventive concepts, as many variations thereof are possible without departing from the spirit and scope of the general inventive concepts. All exemplified amounts are weight percentages based upon the total weight of the composition, unless otherwise specified.

Example 1: The following is an exemplary powdered nutritional composition according to the general inventive concepts.

| | Amount per | |
|---|---|---|
| Ingredient Name | 1000 Kg batch | Kg/g/mg |
| Lactose | 330.4 | kg |
| Nonfat Dry Milk | 202.6 | kg |
| High Oleic Safflower Oil | 117.7 | kg |
| Whey Protein Concentrate | 117.3 | kg |
| Soy Oil | 83.6 | kg |
| Coconut Oil | 81.8 | kg |
| Immunoglobulin | 35.6 | kg |
| Calcium Carbonate | 3.65 | kg |
| Potassium Citrate | 3.49 | kg |
| ARASCO Mortierella alpina Oil | 2.80 | kg |
| Nucleotide-Choline Premix | 2.37 | kg |
| Soy Lecithin | 1.68 | kg |
| Potassium Hydroxide (as solids) (processing | 1.37 | kg |
| aid) | | |
| Ascorbic Acid | 1.29 | kg |
| m-Inositol | 1.29 | kg |
| Vit/Min/Taur Premix | 1.13 | kg |
| Choline Chloride | 1.06 | kg |
| DHASCO Crypthecodinium cohnii Oil | 1.05 | kg |
| Ascorbyl Palmitate | 547.6 | g |
| Carotenoid Premix | 485.0 | g |
| Ferrous Sulfate | 477.4 | g |
| Vitamin A, D3, E, K1 | 368.0 | g |
| Magnesium Chloride | 298.9 | g |
| Potassium Chloride | 267.0 | g |
| Tocopherol-2 Antioxidant | 241.8 | g |
| Sodium Chloride | 36.1 | g |
| L-Carnitine | 8.07 | g |
| Riboflavin | 3.21 | g |
| Copper Sulfate | 2.45 | g |
| Potassium Iodide | 561.5 | mg |
| Tricalcium Phosphate | as needed | |
| Potassium Phosphate Monobasic | as needed | |
| Potassium Hydroxide (processing aid) | as needed | |

Example 2: Bovine immunoglobulin (Ig) was diluted to 50 mg/mL in 37° C. PBS for Adenovirus (AdV) and Rhinovirus (RV) or culture media for Respiratory Syncytial Virus (RSV), rotated in a tube rotator for 0.5-1 hour at ambient temperature, centrifuged at 500-1000×g for 10 minutes at ambient temperature, 0.2 μm syringe filtered and used within 24 hours of preparation.

An appropriate amount of RSV stock for an MOI of 0.1 was combined with Ig to final concentration. Mixtures were incubated for one hour at 37° C. and 5% CO2 then added to confluent HEp-2 cell monolayers in a small volume of infection media. Plates used Ig supplemented infection media to maintain pre-treatment concentration (Ig present pre, during and post infection). After one-hour adsorption at 37° C. and 5% CO₂, inoculum was removed and replaced with infection media with and without Ig supplement. Infected cells were incubated at 37° C. and 5% CO₂ for 2 days then media was harvested for titration.

Virus was diluted at an MOI of 0.1 for RV and 0.5 for AdV in PBS+Ig at target concentration and added to confluent HeLaOH or A549 or cell monolayers. After 1 h adsorption at 33° C. RV or 37° C. and 5% CO₂ for AdV, inoculum was removed and replaced with infection media containing Ig. Cells were incubated for 2 days at 33° C. and 5% CO₂ for hRV and 3 days 37° C. and 5% CO₂ for AdV. Supernatants were collected and frozen for titration.

Antibody stained RSV infected Hep-2 cells at 0.1 MOI incubated at 37° C. and 5% CO₂ for 2 days. HRP-conjugated anti-RSV antibody was used to stain for the presence of RSV while intact cells remain clear.

Cytokine analysis was conducted on adult human peripheral blood mononuclear cells (PBMCs) using a Luminex assay system. Briefly, 50 mL whole blood was collected in sodium heparin tubes and within 2 hours of collection PBMCs isolated using Histopaque for gradient separation. Cells were resuspended at 2 million cells per mL in AIM-V serum free growth media. Resuspended cells were plated at 1 million cells per well in 24 well plates with or without 5 mg/mL bovine immunoglobulin. Cells were incubated for 24 hr at 37° C. and 5% CO₂. Virus was added to an MOI of 1 and incubated for 1 hour at 37° C. and 5% CO₂ for RSV and Adenovirus and 33° C. and 5% CO₂ for Rhinovirus. Fresh media with or without treatment compound was added to a volume of 1 mL and incubated 2 days at 37° C. and 5% CO₂ (33° C. for RV). Cell culture media was harvested and stored at −80° C. until Luminex assay was performed according to manufacturer instructions.

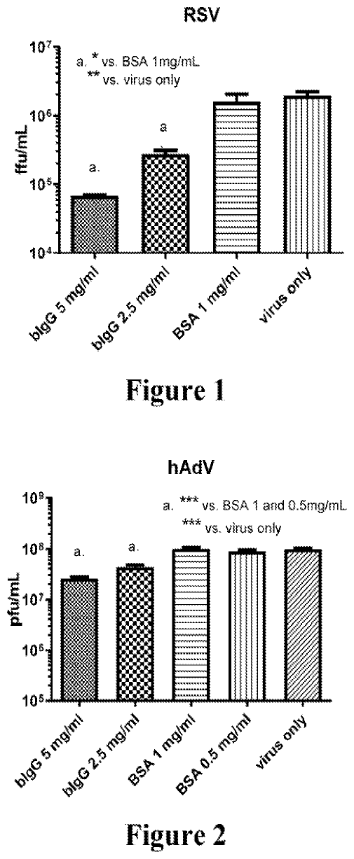

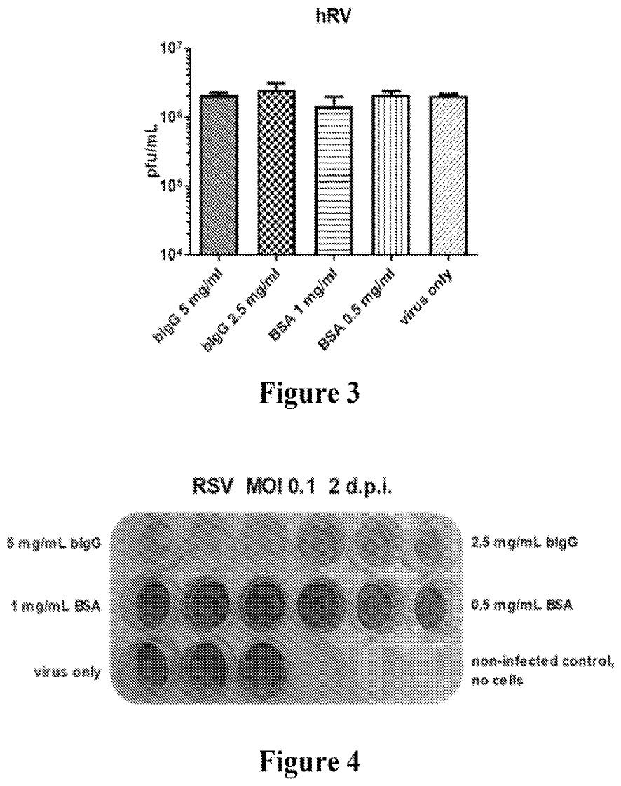

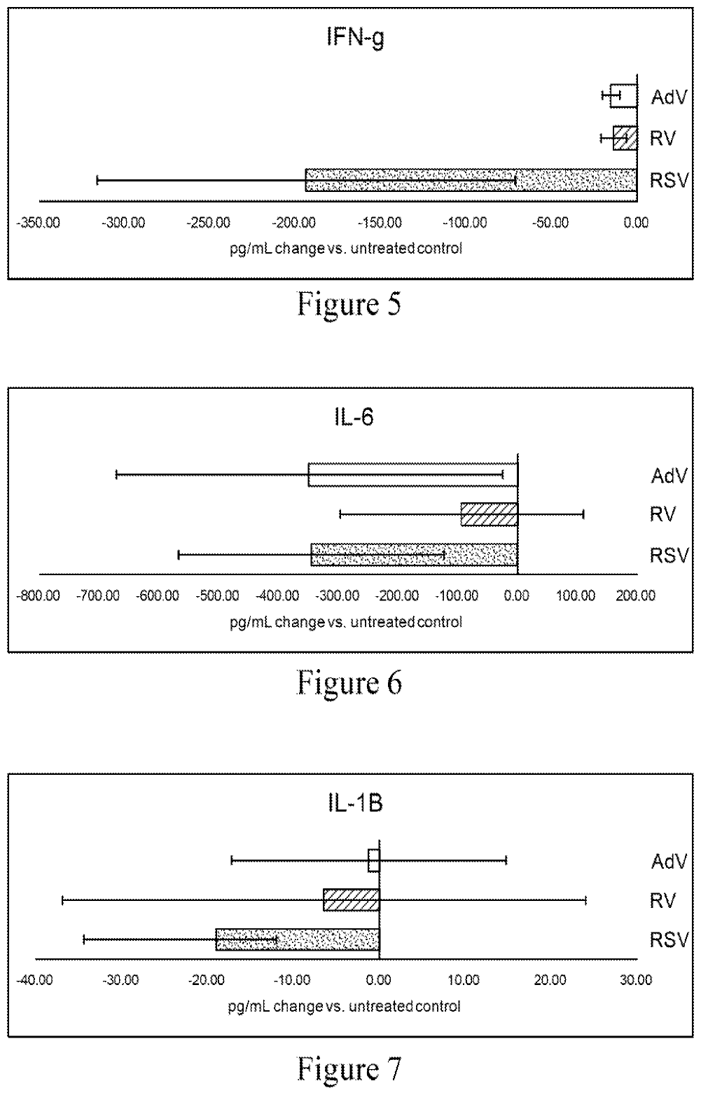

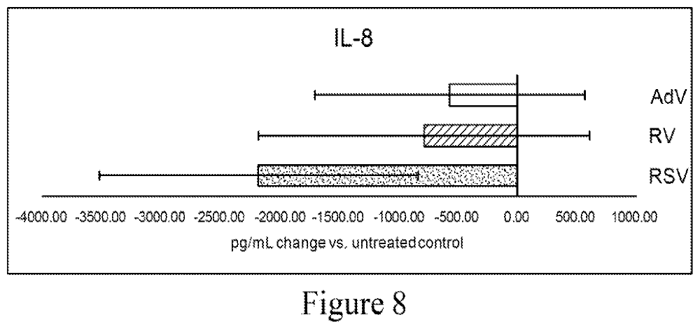

As seen in FIGS. 1-3, Ig reduced viral load in RSV and AdV but not RV infections. However, cytokine levels were reduced dramatically for all three viruses when compared to typical cytokine profiles for the respective viruses (FIGS. 5-8).

Example 3: Human epithelial cell cultures were infected with respiratory syncytial virus (RSV) or rhinovirus (RV16) as described below. Treatments with IgG and IgM were added to cell culture media at the time of infection and maintained throughout incubation. Data are presented as a change in cytokine expression versus virus treated controls.

Methods: Bovine IgG was diluted to 100 mg/mL in infection media and syringe filtered through a 0.2 μm filter. Bovine IgM was first dialyzed in PBS, syringe filtered through a 0.2 μm filter, and then diluted 1:1 with infection media. Materials were adjusted to their final concentrations using infection media. Final concentrations of the bovine antibodies (bAbs) were 5 g/L IgG and 0.55 g/L IgM for the high dose and 2.5 g/L IgG and 0.275 g/L IgM for the lower dose treatments.

For RSV infections, Hep 2 cells were plated in growth media at sufficient concentration to achieve 95% confluence after 24 hours preincubation. Growth media was removed and replaced with OptiMEM containing appropriate concentrations of bAbs. RSV was added to an MOI of 0.1 and incubated at 37 C for 1 hour. After adsorption, final volume was adjusted to 0.5 mL per well with OptiMEM containing appropriate concentrations of bAbs. Cells were incubated for two days after which cell culture media was harvested and stored at −80° C. until Luminex assay was performed according to manufacturer instructions.

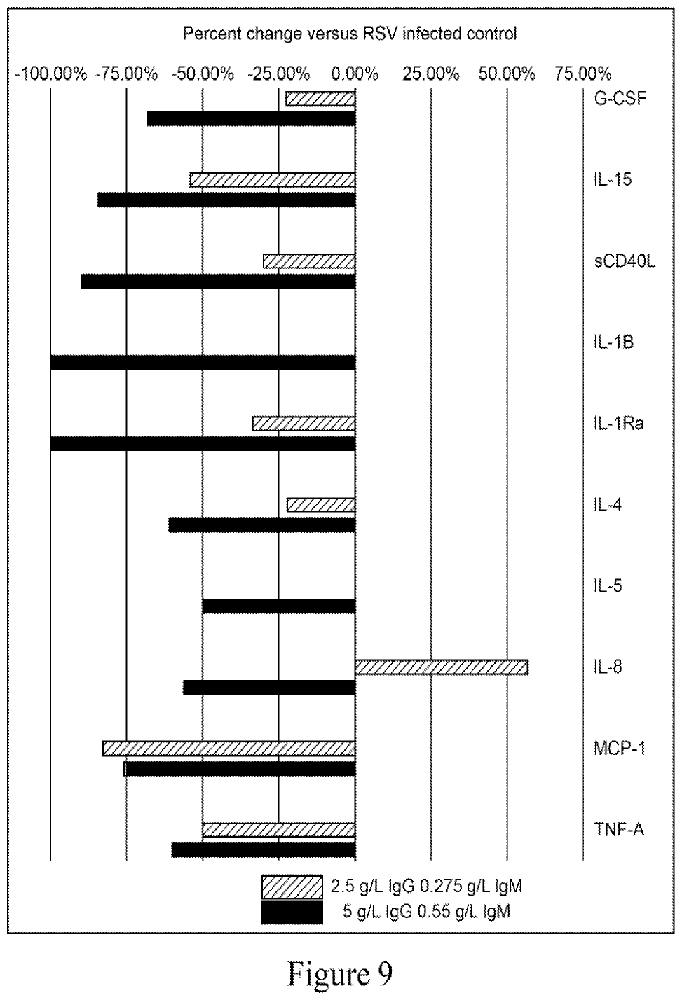

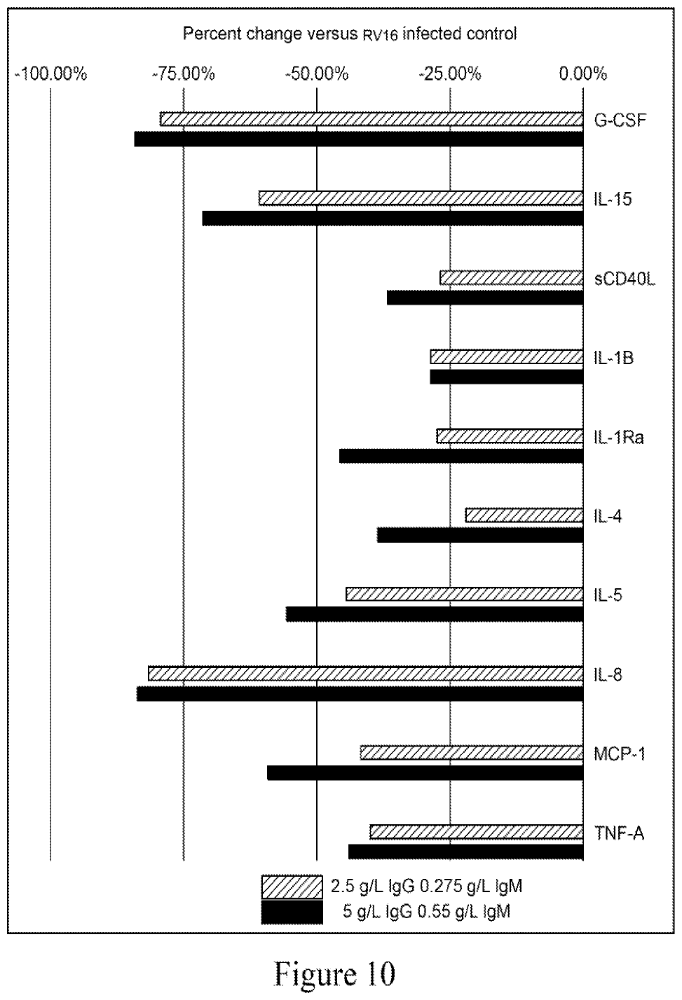

For Rhinovirus16 (RV16) infections, HeLaOH cells were plated in growth media at sufficient concentration to achieve 95% confluence after 24 hours preincubation. Growth media was removed and replaced with infection media containing appropriate concentrations of bAbs. RSV was added to an MOI of 0.05 and incubated at 37 C for 1 hour. After adsorption, the inoculum-containing media was removed and infection media containing bAbs at the appropriate concentrations was added to a volume of 0.5 mL per well and incubated for three days. Cell culture media was harvested and stored at −80° C. until Luminex assay was performed according to manufacturer instructions. The data demonstrate a dose-dependent reduction of several proinflammatory cytokines in the presence of bovine IgG and/or IgM (FIGS. 9 and 10).

Unless otherwise indicated herein, all sub-embodiments and optional embodiments are respective sub-embodiments and optional embodiments to all embodiments described herein. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions or formulations, and illustrative examples shown and described. Accordingly, departures may be made from such details without departing from the spirit or scope of the applicant's general disclosure herein.

1. A method for modulating respiratory virus-induced inflammation in an individual affected by a respiratory virus comprising administering a composition comprising from 0.01 g/L to 100 g/L of bovine immunoglobulin to the individual.

2. The method of claim 1, wherein the composition is nutritional composition which comprises at least one of protein, fat, and carbohydrate.

3. The method of claim 1, wherein the composition is an infant formula.

4. The method of claim 1, wherein the composition is an adult formula.

5. The method of claim 1, wherein the individual is an infirmed adult.

6. (canceled)

7. The method claim 2, wherein the composition comprises lutein.

8. The method claim 2, wherein the composition comprises at least one of vitamin B-6, zinc, and choline.

9. The method claim 2, wherein the inflammation is reduced.

10. The method claim 2, wherein the inflammation is modulated by reducing proinflammatory cytokines.

11. (canceled)

12. The method claim 2, wherein the source of the bovine immunoglobulin has not been hyperimmunized or otherwise intentionally exposed to conditions intended to increase activity against human virus induced inflammation.

13. A composition for treating respiratory virus-induced symptoms, the composition comprising from 0.01 g/L to 100 g/L of bovine immunoglobulin.

14. The composition of claim 13, wherein the composition is a nutritional composition selected from the group of an infant formula, pediatric formula, and an adult formula.

15. The composition of claim 14, wherein the composition is an infant formula.

16. The composition claim 14 further comprising a polyunsaturated fatty acid selected from docosahexaenoic acid, and arachidonic acid, and combinations thereof.

17. The composition claim 14 further comprising lutein.

18. The composition of claim 13, wherein the source of the bovine immunoglobulin has not been hyperimmunized or otherwise intentionally exposed to conditions intended to increase activity against human virus induced inflammation.

19. The composition of claim 13, wherein the composition is a nutritional composition comprising a protein source, a fat source, and a carbohydrate source.

20. The composition of claim 19, further comprising a polyunsaturated fatty acid.

21. The composition of claim 19, wherein the nutritional composition is substantially free of galactooligosaccharides.

22. A method of modulating excessive inflammatory cytokine burden, the method comprising administering a nutritional composition comprising from 0.01 g/L to 100 g/L of bovine immunoglobulin to the individual.

23. The method of claim 22, wherein the inflammation is modulated by modulating proinflammatory cytokines.

24. The method of claim 22, wherein modulating excessive inflammatory cytokine burden decreases sickness/illness behavior symptoms in the individual.

25. The method of claim 22, wherein the source of the bovine immunoglobulin has not been hyperimmunized or otherwise intentionally exposed to conditions intended to increase activity against human virus induced inflammation.

26. The method of claim 24, wherein the sickness/illness behavior symptoms are selected from impaired cognition, fever, anhedonia, loss of appetite, hyperalgesia, and sleep disturbances, lethargy, chills, irritability, skin hypersensitivity to touch.
